(19) United States
(12) Patent Application Publication
(10) Pub. No.: US 2025/0275937 A1
(43) Pub. Date: Sep. 4, 2025

(54) Methods For Reducing Negative Effects Of Alcohol Consumption

(71) Applicant: Honed Life Sciences, LLC, Irvine, CA (US)

(72) Inventor: Avinash Seth, Mission Viejo, CA (US)

(21) Appl. No.: 19/211,755

(22) Filed: May 19, 2025

Related U.S. Application Data
(63) Continuation of application No. 17/521,679, filed on Nov. 8, 2021.
Application No. 17/521,679 is a continuation of application No. 16/363,800, filed on Mar. 25, 2019, now abandoned.
(60) Provisional application No. 62/648,896, filed on Mar. 27, 2018.

(51) Int. Cl. A61K 31/352; A61K 9/20; A61K 31/357; A61K 31/4745; A61K 36/28; A61P 1/16; A61P 25/28

(52) U.S. Cl. CPC A61K 31/352; A61K 9/2009; A61K 9/2013; A61K 9/2054; A61K 31/357; A61K 31/4745; A61K 36/28; A61P 1/16; A61P 25/28

(57) ABSTRACT

Methods for reducing or mitigating concentration problems and dizziness associated with alcohol consumption are described, where a formulation is administered that includes effective amounts of a source of Dihydromycetin (DHM), a source of a milk thistle extract, and a source of Pyrroloquinoline quinone (PQQ) is provided. The source of each DHM and PQQ has at least 95 wt % of the compound. The source of the milk thistle extract has at least 60 wt % silymarin and at least 10 wt % of silybin. The composition is formulated in an orally administrable form consisting of a tablet, a capsule, and an aerosol form. The orally administrable form comprises 10-60 wt % of the DHM, 10-45 wt % of the milk thistle extract, and 0.5-10 wt % of the PQQ in the presence of at least 10 wt % of binder.

This application is a continuation of U.S. patent application Ser. No. 17/521,679, filed Nov. 8, 2021, which is a continuation of U.S. patent application Ser. No. 16/363,800, filed Mar. 25, 2019, which claims priority to U.S. Provisional Application Ser. No. 62/648,896, filed Mar. 27, 2018. This and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling.

### FIELD OF THE INVENTION

The field of the invention is compositions for reducing negative effects associated with alcohol consumption.

### BACKGROUND

The following background discussion includes information that may be useful in understanding the present invention. It is not an admission that any of the information provided herein is prior art or relevant to the presently claimed invention, or that any publication specifically or implicitly referenced is prior art.

Alcohol is a common constituent of medicines, foods, and beverages, which provides both beneficial and detrimental effects on human beings. The term “alcohol” typically refers to ethyl alcohol (ethanol), which is the common form of consumable alcohol found in alcoholic beverages, e.g., such as beer, wine, and liquor. Consumable alcohol is usually produced by fermentation processes of various food products, e.g., including wheat, rice, or other starches, fruits, honey, or other sources of sugars, and yeast.

The National Institute of Alcohol Abuse and Alcoholism (NIAAA) of the U.S. National Institutes of Health (NIH) considers a standard drink to be equal to 0.6 ounces of pure ethanol, e.g., which is equivalent to approximately 12 fluid ounces (fl. oz.) of regular beer (of about 5% alcohol), 8-9 fl. oz. of malt liquor (of about 7% alcohol), 5 fl. oz. of wine (of about 12% alcohol), and 1.5 fl. oz. (referred to as a “shot”) of an 80-proof distilled spirit or liquor (e.g., gin, rum, vodka, whiskey, tequila, etc.)

During consumption, alcohol is rapidly absorbed from the stomach and small intestine into the bloodstream, from which it can affect several organs including the brain, heart, pancreas, and liver. Alcohol can act as a depressant to the central nervous system (CNS). For example, alcohol interferes with the brain's communication pathways, which affects brain functionality that manifests in cognitive and behavioral changes, e.g., such as a person's ability to think, focus, move, as well as his/her mood and behavior. Alcohol can cause inflammation and damage to the liver, e.g., where consistent heavy drinking can cause chronic liver problems. For example, heavy drinking can lead to steatosis (e.g., or fatty liver), infection (e.g., alcoholic hepatitis), fibrosis, cirrhosis, and hepatocarcinoma.

KR20120119863 to Nam Guan Ning teaches various compositions that are reported to reduce damage and side effects associated with alcohol consumption. The compositions comprise acetaldehyde dehydrogenase activator, mitochondrial activator, an antioxidant, and a cell death inhibitor. The cell death inhibitor can cause people to experience digestive disorders such as heartburn, constipation or diarrhea.

U.S. Pat. No. 7,276,514 to Davis teaches various compositions reported to treat and prevent cardiac injury caused by hypoxia or ischemia. The composition includes purified pyrroloquinoline quinone (PQQ). PQQ has been well known to as work as a cardioprotective agent, and it has been found to modulate myocardial oxidative stress, and protect myocardial cells from cell death. PQQ is also well known an anti-oxidant that works by scavenging oxygen radicals from oxidative stress-induced damage, therefore is hypothesized to work to prevent liver damage associated with oxidative stress-induced damage. Experimentation, however, has not shown that the combination of PQQ with other compounds is particularly/synergistically effective to ameliorate the negative effects of excessive alcohol ingestion.

Thus, there is still a need for a composition that effectively ameliorates the negative effects associated with alcohol consumption such as hangover and liver damage, without also causing significant negative side effects.

### SUMMARY OF THE INVENTION

The inventive subject matter provides compositions and methods for reducing negative effects associated with alcohol consumption without causing significant side effects. The negative effects of alcohol consumption include hangovers such as headache, drowsiness, concentration problems, dry mouth, dizziness, fatigue, gastrointestinal distress and nausea, and liver damage such as alcohol-induced hepatic steatosis, hepatitis, hepatic fibrosis, cirrhosis, and hepatocarcinoma. The present inventive composition has been found to reduce or prevent the negative effect of alcohol consumption, and in many cases can be administered prior to, during, or after alcohol consumption.

As of the date of this filing, a preferred drug composition includes dihydromyricetin (DHM), milk thistle extract and pyrroloquinoline quinone (PQQ). DHM, a flavonoid compound from Hovenia, is highly effective in counteracting acute alcohol (EtOH) intoxication, and also withdrawal signs in rats including tolerance, increased anxiety and seizure susceptibility. DHM works by antagonizing acute EtOH-induced potentiation of GABA receptors which are major targets of acute and chronic EtOH actions on the brain. In addition, DHM blocked alcohol-induced GABA expression. (Dihydromyricetin as a novel anti-alcohol intoxication medication, Shen Y, Lindemeyer A K, Gonzalez C, Shao X M, Spigelman I, Olsen R W, Liang J., J Neurosci. 2012 Jan. 4; 32(1):390-401. doi: 10.1523/JNEUROSCI.4639-11). In addition, DHM plays a role to activate alcohol dehydrogenase, such that relieving alcoholic toxicity (KR20120119863A). DHM also exhibits anti-cancer activity against human tumors in vivo and in vitro. It works to reduce reaction oxygen species which are considered to carcinogenesis inducers and to increase expression of proteins contributing to cell apoptosis (A reduction in reactive oxygen species contributes to dihydromyricetin-induced apoptosis in human hepato-cellular carcinoma cells, Liu et al., Sci Rep, Sci, 2014 Nov. 13; 4:7041. doi: 10.1038/srep07041).

Milk thistle (Silybum marianum) is a medicinal plant. The molecule extracted from the seed/fruit of the milk thistle is called silymarin. The extract consists of about 65-80% silymarin. The silymarin was classified by the WHO in the 1970s as an official medicine with hepatoprotective properties. Silymarin is a complex mixture of polyphenolic molecules, including seven closely related flavonolignans (Silybin A, Silybin B, isosilybin A, isosilybin B, silychristin, isosilychristin, silydianin) and one flavonoid (taxifolin). Among these seven compounds, Silybin A and Silybin B are the most active compounds for hepatic diseases. (Silybin, a Major Bioactive Component of Milk Thistle (Silybum marianum L. Gaernt.)-Chemistry, Bioavailability, and Metabolism, Bijak M, Molecules. 2017 Nov. 10; 22(11). pii: E1942. doi: 10.3390/molecules22111942).

PQQ is a natural anti-oxidant which suppresses oxidative stress and liver fibrogenesis in mice and shows a function to prevent and treat liver fibrosis. In addition, cellular oxidative stress and reactive oxygen species are considered to be common inducers of hepato-carcinoma, such that the compound in the present invention may provide the therapeutic effect in hepatocelluar carcinoma. Therefore, the combination of these three molecules is assumed to work as anti-hangover and reducing/preventing the negative effects associated with alcohol consumption.

On surprising result in experimenting with various compositions is that purity of DHM and PQQ appears to significantly affect the results. In preferred embodiments, each of purity of DHM and PQQ is advantageously at least 98% by weight (wt %) and more preferably at least 99% by weight. A similar situation seems to exist with respect to purity of the milk thistle extract, where preferred embodiments have at least 80 wt % silymarin and at least 20 wt % silybin.

Combinations of the three components preferably include at least 10 wt % of DHM, at least 10 wt % of milk thistle and at least 0.5 wt % of PQQ, in the presence of at least 10 wt % of binder. In some embodiments, combinations of the three components preferably include at least 15 wt % of DHM, at least 13 wt % of milk thistle and at least 0.8 wt % of PQQ, in the presence of at least 10 wt % of binder. Especially preferred drug compositions include at least 18 wt % of DHM, at least 16 wt % of milk thistle extract and 1 wt % of PQQ in the presence of at least 10 wt % of binder.

Compositions and methods contemplated herein are preferably provided in an orally administrative capsule, tablet, and/or an aerosol form.

### BRIEF DESCRIPTION OF THE DRAWINGS

FIG. 1 is a chart showing a composition of the formulation in a tablet.

### DETAILED DESCRIPTION

People drink to socialize, celebrate, and relax. However, drinking too much alcohol can lead to suffering such as hangover, liver damage and even death. The current inventive compositions allow people to prevent, or at least significantly reduce, such discomfort and damage in their body, and to relatively quickly return to their normal life.

The compositions herein mainly comprise three active molecules, a) Dihydromycetin (DHM), b) milk thistle extract and c) pyrroloquinoline quinone (PQQ). Some embodiments independently comprise 10-60 wt % DHM, 10-45 wt % milk thistle extract, and 0.5-10 wt % PQQ. Preferred embodiments independently comprise at least 10 wt % DHM, at least 10 wt % milk thistle extract, and at least 0.5 wt % PQQ. More preferred embodiments independently comprise at least 15 wt % DHM, at least 13 wt % milk thistle extract, and at least 0.8 wt % PQQ. The mixture of three molecules above also preferably comprises at least 10 wt % of binder.

The binders include dicalicium phosphate, magnesium stearate, croscarmellose sodium, titanium dioxide, silica, parabens, lactose, maldodextrin, monodosium glutamate, talc, miscrocrystalline cellulose, gelatin, gellan gum, tartrazine, allura red, indigotine, brilliant blue FCF and indigotine, Polyvinylpyrrolidone, and sodium stearyl fumarate, stearic acid.

In some embodiments, the purity of both DHM and PQQ comprises at least 95 wt %, preferably 97 wt %, more preferably 98 wt % and most preferably 99 wt %.

In some embodiments, the milk thistle contains at least 50 wt % of silymarin and at least 5 wt % of Silybins, preferably at least 60 wt % of silymarin and at least 10 wt % of Silybins, preferably at least 70 wt % of silymarin and at least 15 wt % of Silybins, most preferably 80 wt % of silymarin and 20 wt % of Silybins.

Most preferred embodiments independently comprise at least 18 wt % DHM, at least 16 wt % milk thistle extract, and at least 1 wt % PQQ. The mixture of three molecules above also preferably comprises at least 10 wt % of binder.

Presently contemplated compositions are preferably included in an orally administrative tablet, an orally administrative capsule, and/or an aerosol spray to the skin and/or oral such that it can be easily taken prior to, during, or after alcohol consumption.

Tablets/capsules can be produced by any suitable process, for example, molding and/or compression. Tablets/capsules can be produced in any suitable shape that facilitates ingestion, for example, round, ovoid, cylindrical, etc. Similarly, tablets can be produced at any suitable hardness or level of compression as needed to provide suitable cohesion and size.

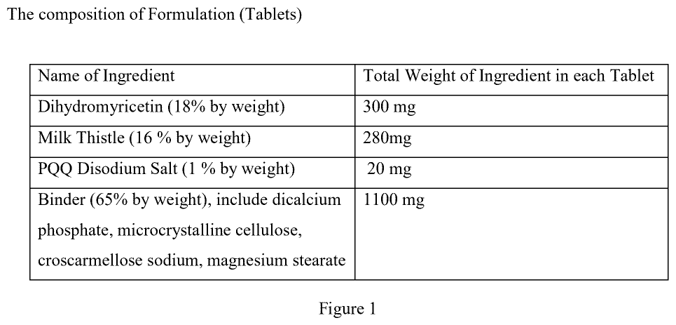

A tablet comprises a composition containing three compounds, DHM, milk thistle, and PQQ. As shown in the FIG. 1, 300 mg of DHM (18 wt %), 280 mg of milk thistle (16 wt %), and 20 mg of PQQ (1 wt %) are contained in 1700 mg of a tablet including 1100 mg (65 wt %) of binders.

### Product Testing

The tablet was examined at least 10 subjects. The subjects consumed two tablets once a day with a glass of water for at least one month prior to significant alcohol consumption (12-16 ounces of hard liquor consumption such as whiskey, rum, gin, and vodka). The result was obtained after alcohol consumption based on a questionnaire described in table 1.

TABLE 1
Ameliorating negative effects of alcohol consumption

| Question | Yes | No | N/A |
|---|---|---|---|
| 1. Is there a reduction in brain fog after | 10 | 0 | 0 |
| consuming 3 tablets within a couple of hours | | | |
| after alcohol consumption | | | |
| 2. Are there any hangover symptoms the | 0 | 10 | 0 |
| following morning (headache, nausea, elevated | | | |
| body temperature etc) after consuming 3 | | | |
| tablets and alcohol consumption the previous | | | |
| evening? | | | |
| 3. Is there a decrease in fatigue and an | 10 | 0 | 0 |
| increase in mental awareness (alacrity) after | | | |
| consuming 3 tables and alcohol consumption? | | | |

Table 1 shows that repeated ingestion of tablets of the test product ameliorates the negative effects of alcohol consumption. For example, all subjects experienced no significant hangover symptoms when they consumed 3 tablets prior to or after significant alcohol consumption. Additional data will be provided to the patent office to show effects relative to controls, and effects relative to use of each of the three active ingredients by themselves, or the three pair combinations.

TABLE 2
Enhanced cognitive abilities

| Question | Yes | No | N/A |
|---|---|---|---|
| 1. Is there a general increase in cognitive | 10 | 0 | 0 |
| abilities (enhanced mental focus and | | | |
| concentration) after consuming 2 tablets on | | | |
| a daily basis (at least 4 weeks) | | | |
| 3. Is there a general increase in cognitive | 9 | 0 | 1 |
| abilities (enhanced memory, reasoning, and | | | |
| analytical abilities) after consuming 2 | | | |
| tablets daily basis (at least 4 weeks) | | | |

Table 2 shows that ingestion of tablets of the test product plays a role in enhancing cognitive abilities when the subjects took 2 tablets every day for at least 4 weeks prior to the questionnaire. This was an unexpected result because none of the three compounds by themselves shows such an effect in the prior art. The enhanced cognitive abilities seem only to be observed in conjunction with ingestion of the combination of the three compounds. Here again, additional data will be provided to the patent office to show effects relative to controls, and effects relative to the use of each of the three active ingredients by themselves, or the three pair combinations.

The cognitive abilities in the questionnaire include memory, attention, and executive functions. Further studies will be conducted to assess changes in perception, motor skills, language skill, and visual and spatial processing, as well as possible side effects. The results will be provided to the patent office.

A reduction of alcohol-induced hepatic diseases such as hepatic steatosis, hepatitis, hepatic fibrosis, cirrhosis, and hepatocarcinoma will be examined by the use of the composition. The results will be provided to the patent office.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method for reducing or mitigating a concentration problem associated with alcohol consumption, comprising:
administering a formulation comprising a source of Dihydromycetin (DHM), a source of silymarin, and a source of Pyrroloquinoline quinone (PQQ) to the individual in need of treatment,
wherein the formulation is provided in an amount effective to reduce or mitigate the concentration problem associated with alcohol consumption.

2. A method for reducing or mitigating dizziness associated with alcohol consumption, comprising:
administering a formulation comprising a source of Dihydromycetin (DHM), a source of silymarin, and a source of Pyrroloquinoline quinone (PQQ) to the individual in need of treatment,
wherein the formulation is provided in an amount effective to reduce or mitigate dizziness associated with alcohol consumption

3. The method of claim 1 or 2, wherein the source of silymarin has at least 60 wt % silymarin and at least 10 wt % silybin.

4. The method of claim 1 or 2, wherein the formulation is in an orally administrable form selected from the group consisting of a tablet, a capsule, and an aerosol form.

5. The method of claim 4, wherein the tablet includes at least 10% of the DHM by weight.

6. The method of claim 4, wherein the tablet includes at least 10% of the milk thistle extract by weight.

7. The method of claim 4, wherein the tablet includes at least 0.5% of the PQQ by weight.

8. The method of claim 4, wherein the tablet comprises at least 10% of binder by weight.

9. The method of claim 4, wherein the tablet comprises 10% to 60% by weight of the DHM, 10% to 45% by weight of the milk thistle extract, and 0.5% to 10% by weight of the PQQ.

10. The method of claim 4, wherein the tablet comprises 18% by weight of the DHM, 16% by weight of the milk thistle extract, and 1% by weight of the PQQ.
